(19) United States
(12) Patent Application Publication
(10) Pub. No.: US 2025/0366735 A1
(43) Pub. Date: Dec. 4, 2025

(54) Wearable Sensor Assembly for Early Detection of Plantar Fasciitis

(71) Applicant: Anaya Bera, MILPITAS, CA (US)

(72) Inventor: Anaya Bera, MILPITAS, CA (US)

(21) Appl. No.: 19/223,056

(22) Filed: May 30, 2025

Related U.S. Application Data
(60) Provisional application No. 63/654,111, filed on May 31, 2024.

(51) Int. Cl. A61B 5/107; A61B 5/00

(52) U.S. Cl. CPC A61B 5/1071; A61B 5/1074; A61B 5/6829; A61B 5/7275; A61B 5/742; A61B 2560/0462; A61B 2562/227

(57) ABSTRACT

One or more embodiments of the invention are a novel wearable device assembly that help in early detection of plantar fasciitis and improve mobility. Particularly, the present invention relates to a wearable sensor assembly which detects calcaneal inclination (pitch) angle using flex sensor that determines risk factor of plantar fasciitis. This invention functions by measuring the calcaneal pitch angle of patient's foot and comparing that with the normal calcaneal pitch angle established for normal foot. The risk factor of plantar fasciitis is predicted as calcaneal pitch angle of the foot is significantly lower in patients with plantar fasciitis than in normal subjects. With an early diagnosis of plantar fasciitis, patients can receive early treatment leading to improved mobility and better quality of life. This wearable sensor can work wirelessly and enable real-time data collection from the patients to assist medical professionals during the detection and treatment of plantar fasciitis.

### FIELD OF THE INVENTION

The invention is related to a non-invasive wearable sensor assembly for early detection of plantar fasciitis (PF) and improving mobility. Particularly, the present invention relates to a wearable sensing device which detects calcaneal inclination (pitch) angle using flex sensor that determines risk factor of plantar fasciitis. Plantar fasciitis affects about 10% of the population and reduces mobility. This invention functions by measuring the calcaneal inclination angle of patients feeling heel pain and comparing the normal calcaneal inclination angle established for normal foot. The risk factor of plantar fasciitis can be predicted as calcaneal inclination angle is significantly lower in patients with plantar fasciitis than in normal subjects. With an early diagnosis of plantar fasciitis, patients can receive early treatment leading to improved mobility and better quality of life. This wearable sensor can work wirelessly and enable real-time data collection from the patients to assist medical professionals during the detection and treatment process.

### BACKGROUND

Plantar fasciitis occurs due to the inflammation of plantar fascia. The plantar fascia is the tissue (ligament) that connects the heel bone (calcaneal tuberosity) to the base of the toe. The plantar fascia supports the arch of the foot and absorbs shock when walking. Repeated stretching and tearing of the tissue can cause inflammation of the plantar fascia.

Plantar fasciitis is the most common cause of heel pain. Plantar fasciitis affects about 10% of the population and reduces mobility. Plantar fasciitis arises from the degenerative irritation of the plantar fascia originating at the medial calcaneal tuberosity of the heel and its surrounding perifascial structures.

Plantar fasciitis is multifactorial in etiology. Intrinsic factors include age, obesity, excessive foot pronation, and limited ankle dorsiflexion. Extrinsic factors include inappropriate shoe wear, occupational prolonged weight-bearing, and rapid increase in activity level. These factors combine to develop a pathologic overload of the plantar fascia at the calcaneal insertion, causing microtears in the fascia that subsequently led to plantar fasciitis. As small tears within the fascia increase in size and become symptomatic, the gait cycle alters the normal biomechanics of the foot, creating mobility impairments.

The current diagnosis methods for plantar fasciitis include X-Ray, CT scan, Ultrasound and MRI Scan that are expensive. The diagnosis using pain scale 1-10 is subjective. Therefore, there is a need in the art for diagnosis of plantar fasciitis that is inexpensive, quantitative, and not subjective.

### DESCRIPTION OF THE DRAWINGS

It is to be noted that the appended drawings illustrate only typical embodiments of this disclosure and are therefore not to be considered limiting of its scope, for the disclosure may admit to other equally effective embodiments.

FIG. 1 shows a bottom view of plantar side of a dissected human foot including plantar fascia without plantar fasciitis.

FIG. 2 shows a bottom view of plantar side of a dissected human foot including plantar fascia with plantar fasciitis.

FIG. 3 shows a side view of a dissected human foot including calcaneal inclination (pitch) angle.

FIG. 4 shows a wearable device assembly for detecting plantar fasciitis as a system in accordance with one or more embodiments of the present invention.

FIG. 4A shows a wearable device assembly for detecting plantar fasciitis as a system using serial connection in accordance with one or more embodiments of the present invention.

FIG. 4B shows a wearable device assembly for detecting plantar fasciitis as a system with wireless connection in accordance with one or more embodiments of the present invention.

FIG. 5A shows a flexible wearable device assembly comprising flexible sensor and socks in accordance with one or more embodiments of the present invention.

FIG. 5B shows a flexible wearable device assembly comprising flexible sensor and splint in accordance with one or more embodiments of the present invention.

FIG. 6 shows a flex sensor whose electrical resistance depends on the magnitude of a bend or flex angle of the flex sensor.

### DETAILED DESCRIPTION OF THE INVENTION

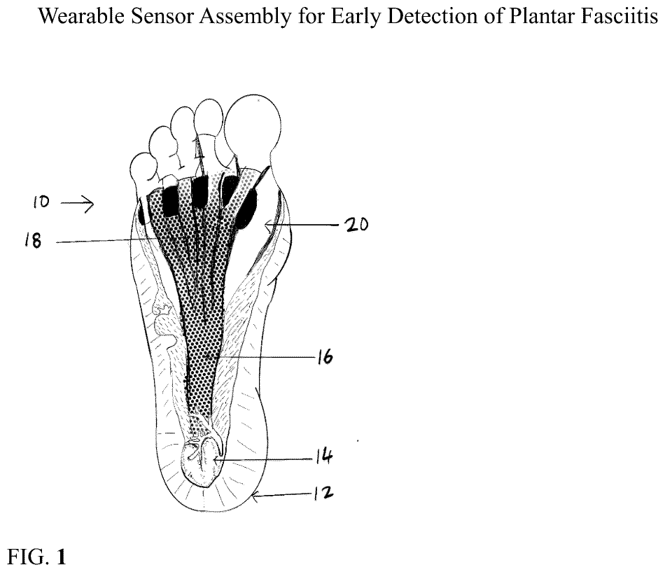

FIG. 1 shows a typical dissected bottom view of the plantar side of a human foot 10 including a heel region 12 having a heel bone 14, superficial tracks, 18 and a ball region 20. The foot includes a plantar fascia 16 that extends across the plantar side of the foot 10 from the heel region 12 to the ball region 20. Plantar fascia 16 serves a vital role in maintaining the shape of the two anatomical arches of the foot, the transverse arch, and the longitudinal arch. When an individual walks, the plantar fascia 16 functions as a shock absorber and transfer tension forces over the plantar side of the foot 10.

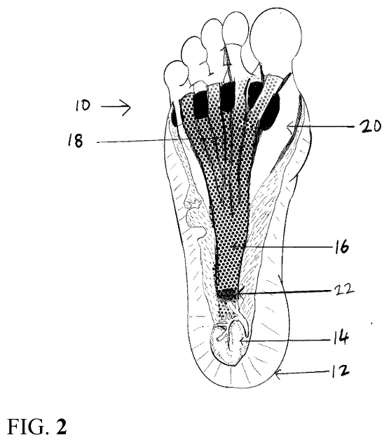

Overstressing the plantar fascia 16 may produce tears in the plantar fascia or separate the plantar fascia from the heel bone 14 and other surrounding tissues of the foot 10. Tearing and separation of the plantar fascia 16 produces painful inflammation commonly referred to as plantar fasciitis. In FIG. 2, the inflamed area 22 of the plantar fascia 16 is located near the heel bone 14 in the heel region 12 of the foot 10. In other instances, the pain from plantar fascia may be felt along the arch region of the foot 10.

Left untreated, plantar fasciitis may become very debilitating so that common activities such as walking and standing are very painful. Common non-surgical treatments for plantar fasciitis may include bed rest, stretching the plantar fascia, applying ice packs to the plantar side of the feet, using night splints and wedge-shaped arch supports, ingesting oral anti-inflammatories, and receiving steroid injections. Non-surgical treatments have not proven to be very successful. In some instances, treating chronic, severe cases of plantar fasciitis may require corrective surgery on the plantar fascia.

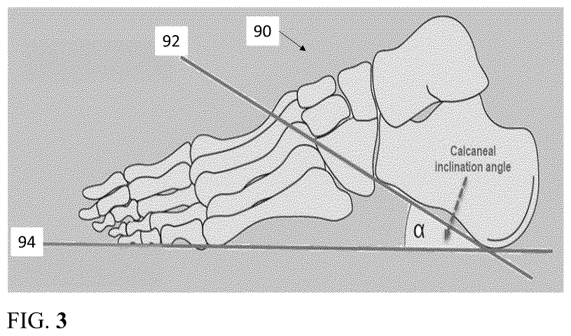

The side view of human foot bone, 90, in FIG. 3 shows plantar plane, 94 and line tangent to interior calcaneal border, 92. The angle, a, between the plantar plane, 94 and line tangent to interior calcaneal border, 92 is defined as calcaneal inclination (pitch) angle.

The invention is related to quantitative measurement of calcaneal inclination angle, a, using biomechanical assessment for early detection of plantar fasciitis.

There are no methods currently available that detect plantar fasciitis using the measurement of calcaneal inclination angle. The measured calcaneal inclination angle and risk factors can be transferred using the serial port locally or transmitted wirelessly to remote location for assessment by medical professionals to detect plantar fasciitis.

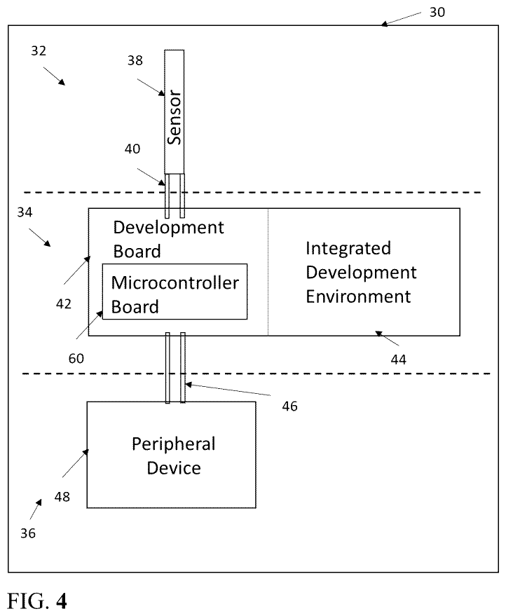

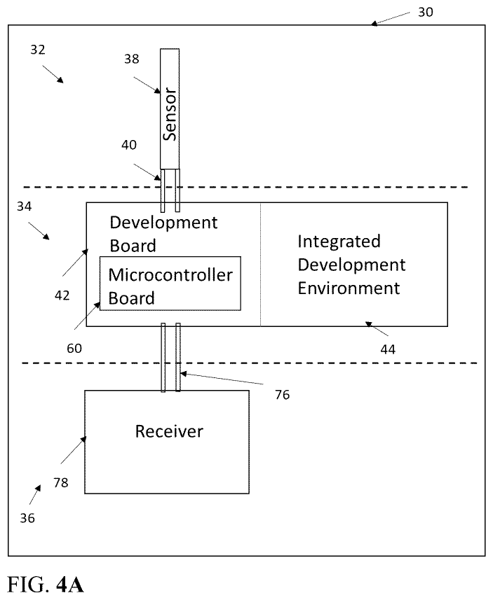

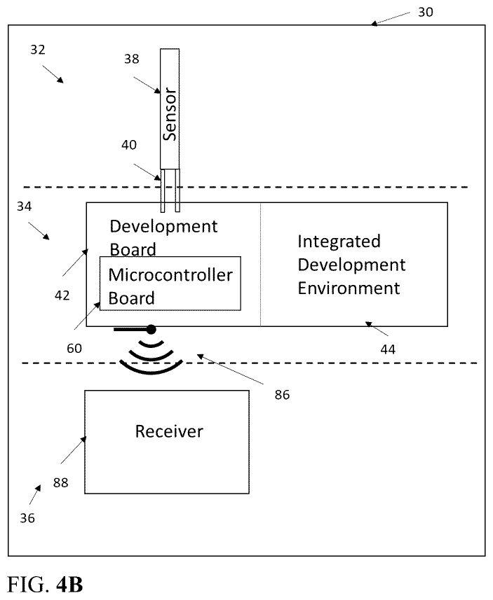

As shown in FIG. 4, A wearable device assembly, 30 for detecting plantar fasciitis as a system is comprised of an input system, 32, a computational system, 34, and an output system, 36. The input system, 32, is composed of a sensing device or sensor particularly a flex sensor, 38, for measuring angle dependent resistance of the sensor based on flex angle. An example of flex sensor is Flex Sensor by Spectra Symbol. The computational system, 34 comprises a development board, 42 and an integrated development environment, 44. The development board includes a microcontroller board, 60 with connectors for organized wires. Examples of microcontroller board, 60 are Arduino, Raspberry Pi, etc.

The development board, 42 as in FIG. 4 connects to the sensor, 38 in the input system, 32 using organized wire, 40, and to the peripheral device, 48, in the output system, 36 using the connector, 46. The microcontroller board, 60, as part of development board, 42, translates resistance of the sensor, 38 into calcaneal inclination (pitch) angle, a and corresponding risk factor. A calcaneal pitch angle, α<18° is considered high risk, and an angle, α>=18° is considered low or no risk for plantar fasciitis. The data of calcaneal pitch angle, α and risk factor of plantar fasciitis are calculated in the development board, 42, and sent to the output system, 36 through the connector, 46. The output system consists of a peripheral device, 48. The data can be displayed, streamed and/or stored in the peripheral device, 48.

As shown in FIG. 4A, A wearable device assembly, 30 for detecting plantar fasciitis as a system is comprised of an input system, 32, a computational system, 34, and an output system, 36. The input system, 32, is composed of a sensing device or sensor particularly a flex sensor, 38, for determining angle dependent resistance based on flex angle. The computational system, 34 comprises a development board, 42 and an integrated development environment, 44. The development board includes microcontroller board, 60, and a serial connector, 76 to the receiver, 78. The data of calcaneal pitch angle, a and risk factor of plantar fasciitis are calculated in the development board, 42, and sent to the output system, 36 through the serial connector, 76 using a serial port. The serial connector, 76 can be a universal serial bus. The output system consists of a receiver, 78. The receiver can be a serial monitor or a spreadsheet. The data can be displayed, streamed and/or stored in the receiver, 78, using an application.

As shown in FIG. 4B, A wearable device assembly, 30 for detecting plantar fasciitis as a system is comprised of an input system, 32, a computational system, 34, and an output system, 36. The input system, 32, is composed of a sensing device or sensor particularly a flex sensor, 38, for determining angle dependent resistance based on flex angle. The computational system, 34 comprises a development board, 42 and an integrated development environment, 44. The development board includes microcontroller board, 60 and a wireless device, 86 to the receiver, 88. The data of calcaneal pitch angle, a and risk factor of plantar fasciitis are calculated in the development board, 42, and sent to the output system, 36 wirelessly through the Wi-Fi or Bluetooth connection, 86. Examples of Bluetooth transmitter can be HC06, HC05, etc. The output system consists of a receiver, 88. such as smart phones, laptops, etc. The data can be displayed, streamed and/or stored in the receiver, 88 using an application. An example of an application is MIT App Inventor.

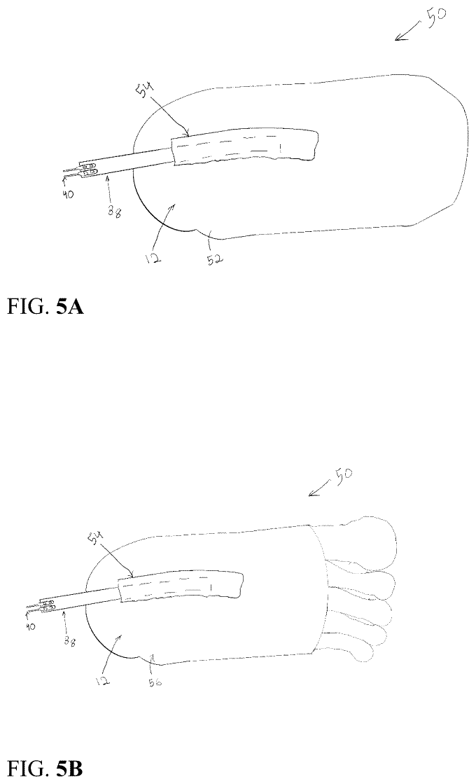

The sensing device or flex sensor, 38, is located inside a pocket, 54 of a wearable device, such as socks, 52, worn on a foot of a human, made of flexible material. The flexible wearable device assembly, 50 in FIG. 5A, is suitable for positioning near the center of the heel region, 12 and extended several inches into the longitudinal arch of a patient's foot along the plantar fascia, 16 as in FIG. 1.

The sensing device or flex sensor, 38, is located on a wearable device, such as splint, 56, worn on a foot of a human, made of flexible material, of the flexible wearable device assembly, 50 in FIG. 5B, suitable for positioning near the center of the heel region, 12, and extended several inches into the longitudinal arch of a patient's foot along the plantar fascia, 16 as in FIG. 1.

Examples of flexible materials can be nylon, polyester, neoprene, etc. The flex sensors, 38, can be non-invasive. The length of the flex sensor can range from 2 inches to 5 inches. An example of flex sensor is Flex Sensor by Spectra Symbol.

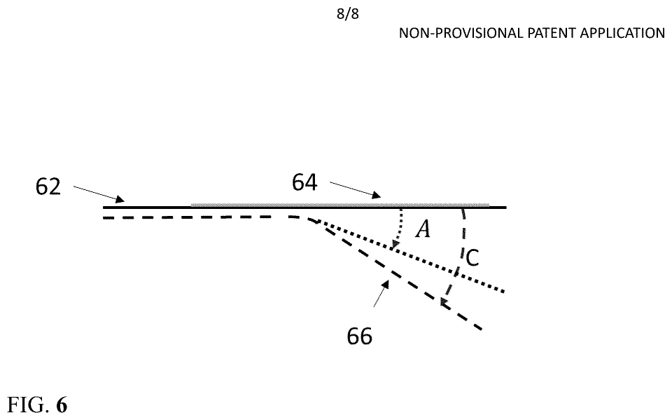

The flex sensor, 62 as in FIG. 6 comprising an elongated body of a material whose electrical resistance, R_{A} depends on a magnitude of a flex or bend angle A of the elongated body. The change in resistance of the conductive ink, 64 with bend angle A determines the resistance of flex sensor, R_{A}. When the flex sensor is flat at position 62 with an angle of 0°, the flat resistance is R_{F}. The flat resistance can range from several thousands (e.g., 7,000 Ohm) to tens of thousands (e.g., 50,000 Ohm). When the flex sensor is bent by a defined angle C, the resistance is R_{C}. The resistances R_{F} and R_{C} are measured using a multimeter to calibrate the flex sensor, 38. The corresponding angles are measured using a protractor. The resistance of the flex sensor, R_{A}, for a flex angle, A is interpolated as

$${R}_{A} = {R}_{F} + \frac{A ( {R}_{C} - {R}_{F} )}{C}$$

During the diagnosis, the flexing (bending) of the angle of the flex sensor on a patient's foot leads to the resistance R_{α} of the sensor. Based on the resistance R_{α} calcaneal inclination (pitch) angle, α is computed using the microcontroller.

$$α = \frac{C ( {R}_{α} - {R}_{F} )}{( {R}_{C} - {R}_{F} )}$$

The computational system, 34 comprises a development board, 42 and an integrated development environment, 44 as in FIG. 4. The development board includes a microcontroller board, 60, and a connector, 46 to the peripheral device, 48. The development board, 42 connects to the sensor, 38 for resistance data input using the organized wires, 40. The microcontroller perform computation of calcaneal inclination (pitch) angle, α based on resistance, R_{A} of the flex sensor, 38. The calcaneal inclination angle, α determines the risk factor of plantar fasciitis. A calcaneal pitch angle, α<18° is considered high risk, and an angle, α>=18° is considered low or no risk for plantar fasciitis. The data of calcaneal pitch angle, α and risk factor of plantar fasciitis are sent to the output system, 36 through the connector, 46.

The flexible sensor can be embedded or weaved into the socks or splints using e-textile. This can be used for medical monitoring, and/or early detection of plantar fasciitis.

The present method involves the measurement of flex angle and determining the angular deviation comparing with normal subject using bio-mechanical assessment. The methodology is not limited to plantar fasciitis and can be used for detecting deformity such as abnormal posture.

The device and methodology in early detection of plantar fasciitis can be used beyond human care, such as animal care.

While the foregoing is directed to embodiments of the present disclosure, other and further embodiments of the disclosure may be devised without departing from the basic scope thereof, and the scope thereof is determined by the claims that follow.

What is claimed is:

1. A wearable device assembly for detecting plantar fasciitis as a system, comprising: an input system composed of sensing device(s) for determining angle dependent resistance based on flex angle; a computational system for translating resistance into calcaneal inclination (pitch) angle and corresponding risk factor; and an output system that streams or displays calcaneal inclination angle and/or risk factor to peripheral device(s).

2. The wearable device of claim 1, wherein the sensing devices(s) are located on a flexible material suitable for positioning near the center of the heel bone (calcaneal tuberosity) and extended several inches into the medial longitudinal arch of a patient's foot.

3. The wearable device of claim 1, wherein the sensing devices(s) are non-invasive.

4. The wearable device of claim 1, wherein the computational system comprises a development board, and an integrated development environment.

5. The computational system in claim 4, wherein the development board comprises a microcontroller board and connectors for organized wires.

6. The computational system in claim 4, wherein the development board includes microcontroller board, and a wireless device to transmit data.

7. The computational system of claim 4, wherein the development board connects to the sensing device for data collection using the organized wires.

8. The computational system of claim 4, wherein the integrated development environment computes calcaneal inclination (pitch) angle and corresponding risk factor based on the angle.

9. The calcaneal inclination (pitch) angle in claim 8 can be from 0° to 32°. For the calcaneal inclination (pitch) angle less than 18°, the risk factor for plantar fasciitis is high. For the calcaneal inclination (pitch) greater than or equal to 18°, the risk factor for plantar fasciitis is low.

10. The wearable output system of claim 1 wherein the peripheral device is either attached to or remote from the computational system that streams or displays calcaneal inclination angle and/or risk factor.

11. The attached peripheral device to the computational system in claim 10 can be a serial monitor or a secured digital card or other memory devices for data streaming, display and/or storage.

12. The remote peripheral device to the computational system in claim 10 can be a smart phone, tablet or similar device(s) with applications connected using Bluetooth, Wi-Fi or other wireless communication protocol for data streaming, display and/or storage through a wireless module attached to the development board.
